(19) United States
(12) Patent Application Publication
(10) Pub. No.: US 2010/0168247 A1
(43) Pub. Date: Jul. 1, 2010

(54) SOLID COMPOSITES OF A CALCIUM RECEPTOR-ACTIVE COMPOUND

(75) Inventors: Ilan Zalit, Rosh Ha Ayin (IL); Uri Zadok, Herzliyya (IL)

Correspondence Address: KENYON & KENYON LLP, ONE BROADWAY, NEW YORK, NY, 10004, US

(73) Assignee: TEVA PHARMACEUTICAL INDUSTRIES LTD., Petah Tiqva (IL)

(21) Appl. No.: 12/723,268

(22) Filed: Mar. 12, 2010

Related U.S. Application Data
(62) Division of application No. 11/897,684, filed on Aug. 30, 2007.
(60) Provisional application No. 60/841,689, filed on Sep. 1, 2006.

(51) Int. Cl. A61K 31/137; A61P 5/18

(52) U.S. Cl. 514/655

(57) ABSTRACT

The invention encompasses solid composites of the calcium receptor-active compounds, processes for preparing the solid composites, immediate and controlled-release pharmaceutical formulations comprising the solid composites, and methods of treatment therewith.

### CROSS-REFERENCE TO RELATED APPLICATIONS

This application is a divisional of U.S. application Ser. No. 11/897,684, filed Aug. 30, 2007, which claims the benefit of U.S. Provisional Application Ser. No. 60/841,689, filed Sep. 1, 2006, which is incorporated herein by reference in its entirety.

### FIELD OF THE INVENTION

The invention encompasses solid composites of the calcium receptor-active compounds, processes for preparing the solid composites, immediate and controlled-release pharmaceutical formulations comprising the solid composites, and methods of treatment therewith.

### BACKGROUND OF THE INVENTION

Cinacalcet hydrochloride has the chemical name (R)—N-[1-(1-naphthyl)ethyl]-3-[3-(trifluoromethyl)phenyl]propan-1-amine hydrochloride and is reported to have the following chemical structure:

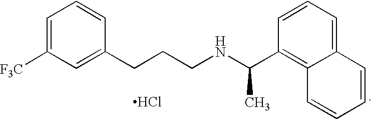

Cinacalcet hydrochloride is a calcium receptor-active compound that is currently marketed under the trade name SENSIPAR® for the treatment of hyperparathyroidism in patients with chronic kidney disease on hemodialysis. Physicians' Desk Reference, 60th ed. (2006), pp. 603-605. The dosing of SENSIPAR® is expressed in terms of amount of cinacalcet free base, (R)—N-[1-(1-naphthyl)ethyl]-3[3-(trifluoromethyl)phenyl]propan-1-amine, present in the tablet, rather than in terms of the amount of the hydrochloride salt. See id.

Cinacalcet is a solid that is understood to be slightly soluble in water and very soluble in some organic solvents, such as methanol and ethanol. For example, U.S. patent application Ser. No. 10/937,870, published as U.S. Patent Application Publication No. 2005/0147669 (“the '669 publication”) reports that cinacalcet has a solubility in water of less than about 1 μg/ml at neutral pH. The '669 publication, p. 1, ¶ 2. Further, the '669 publication reports that the solubility of cinacalcet can reach about 1.6 mg/ml when the pH ranges from about 3 to about 5. However, when the pH is about 1, the solubility decreases to about 0.1 mg/ml. Id.

Compounds having low water solubility typically demonstrate a low rate of dissolution and, often, low bioavailability. See, e.g., Ansel, et al., Pharmaceutical Dosage Forms and Delivery Methods (6th ed., 1995), pp. 105, 108.

The '669 publication addresses the poor water solubility of cinacalcet by providing pharmaceutical compositions comprising cinacalcet in the form of particles with a D₅₀ of less than or equal to about 50 μm. '669 publication, pp. 2-3, ¶ 26. The '669 publication discloses that these pharmaceutical compositions have a dissolution profile that results in about 50 percent to about 125 percent of a target amount of cinacalcet being released from the composition no later than about 30 minutes from the start of a dissolution test that is conducted in 0.05 N HCl in a U.S.P. type 2 apparatus at a temperature of 37° C.±0.5° C. at a rotation speed of 75 r.p.m. Id. at p. 5, ¶ 61.

However, this technique of increasing the solubility in water, and, thus, the bioavailability, of cinacalcet requires micronization of the cinacalcet to achieve the desired particle size distribution. Micronization poses a health risk during production on an industrial scale. Thus, there is a need for a method to reduce the health risks involved with micronized powders of active ingredients, such as cinacalcet, while maintaining an adequate dissolution profile, and, thus, bioavailability.

There is also a need for a method of releasing drugs, such as cinacalcet, at a position in the gastrointestinal tract where the pH is such that they are most soluble.

### SUMMARY OF THE INVENTION

The present invention is directed to a composition, comprising a solid composite of cinacalcet in intimate association with at least one carrier. In preferred embodiments of the invention, the composition is the solid composite. In preferred embodiments, at least about 85 percent of the cinacalcet is in intimate association with the at least one carrier, at least about 85 percent of the cinacalcet is not in particulate form, at least about 85 percent of the cinacalcet is not in crystalline form, and/or the solid composite is a solid solution. Preferably, substantially all of the cinacalcet is in solution in the solid solution.

In preferred embodiments of the invention, the carrier comprises a polymer, such as povidone, poloxamer, hydroxypropyl methylcellulose, polyethylene glycol, copovidone, copolymers of methacrylate, copolymers of methacrylic acid, and mixtures thereof. Preferably, the polymer is povidone or a copolymer of methacrylic acid. In preferred embodiments the carrier comprises a sugar or sugar derivative, such as sucrose, mannitol, lactose, maltitol, sorbitol, xylitol, sucralose, and mixtures thereof.

In preferred embodiments of the invention, the solid composite is a particulate having an average particle size of more than about 100 μm, preferably, from about 100 to about 600 μm. In preferred embodiments of the invention, the composite has a drug to carrier weight ratio of from about 1:0.5 to about 1:10, preferably, from about 1:2 to about 1:6.

In preferred embodiments of the invention, the composition is a pharmaceutical formulation, comprising from about 10 percent to about 40 percent by weight of cinacalcet. In preferred embodiments of the invention, the composition further comprises about 0.5 percent to about 5 percent by weight relative to the total weight of the formulation of at least one glidant or lubricant and/or about 1 percent to about 6 percent by weight of at least one coating material.

In preferred embodiments of the invention, the composition is an immediate release composition from which at least about 80 percent of the cinacalcet is released within about 30 minutes in 0.05 N HCl in a U.S.P. type 2 apparatus at a temperature of about 37° C. and at a rotation speed of about 75 r.p.m.

In preferred embodiments of the invention, the composition is a controlled release pharmaceutical formulation, and wherein, when the formulation is exposed to a solution of 0.05 N HCl in a U.S.P. type 2 apparatus at a temperature of about 37° C. and at a rotation speed of about 75 r.p.m. for about 30 minutes, followed by addition of a buffer in an amount sufficient to neutralize the solution and continued exposure to the neutralized solution, more than about 50 percent of the cinacalcet is released from the formulation within about the first 30 minutes of exposure, and not less than about 70 percent of the calcium receptor-active compound is released from the formulation within about the first 90 minutes of exposure. Preferably, at least about 50 percent of the cinacalcet is released within about the first 60 minutes of exposure, more preferably, not less than about 80 percent of the cinacalcet is released from the formulation during about the first 90 minutes of exposure, and, most preferably, not less than about 90 percent of the cinacalcet is released from the formulation during about the first 90 minutes of exposure.

In preferred embodiments of the invention, the invention is directed to a method for preparing a solid composite, comprising combining cinacalcet, at least one carrier, and at least one liquid solvent to form a solution, and removing the solvent to obtain a solid composite of the cinacalcet and the at least one carrier. In preferred embodiments of the invention, the solid composite is a solid solution. In preferred embodiments of the invention, the carrier is selected from the group consisting of povidone, poloxamer, hydroxypropyl methylcellulose, polyethylene glycol, copovidone, copolymers of methacrylate, and copolymers of methacrylic acid, preferably at least one of lower aliphatic alcohols and C₃₋₈ ketones. In preferred embodiments of the invention, the solvent is removed by evaporation, preferably under vacuum, in a fluidized bed drier, or by spray drying. In preferred embodiments of the invention, the composition is the cinacalcet and at least one carrier are dissolved in an organic or inorganic solvent to form the solution, a supercritical fluid is added to induce precipitation of a mixture of the cinacalcet and the carrier, and the solvent and the supercritical fluid are removed by evaporation. In preferred embodiments of the invention, the solvent is a supercritical fluid, preferably, at least one of carbon dioxide, water, methane, ethane, propane, ethylene, propylene, methanol, ethanol, and acetone, and, more preferably, carbon dioxide. In preferred embodiments of the invention, the supercritical fluid is removed by evaporation.

In preferred embodiments, the invention the invention is directed to a method for preparing the solid composite, comprising combining cinacalcet and at least one carrier to form a mixture, heating the mixture to a temperature at which both the cinacalcet and the carrier melt to form a fusion product, and cooling the fusion product in a manner that does not allow crystallization of the cinacalcet out of the fusion product. In preferred embodiments of the invention, the method further comprises combining the cinacalcet and at least one carrier to form a mixture, heating the mixture to control its viscosity, and feeding the heated mixture through a hot melt extrusion system.

In preferred embodiments the invention, is directed to a method of treatment, comprising administering an effective amount of a preferred composition of the invention to a mammal, such as a human.

### BRIEF DESCRIPTION OF THE DRAWINGS

FIG. 1 illustrates an XRD diffractogram of cinacalcet raw material.

FIG. 2 illustrates an XRD diffractogram of PVP K-30.

FIG. 3 illustrates an XRD diffractogram of a 1:2 solid solution of cinacalcet with PVP K-30.

FIG. 4 illustrates an XRD diffractogram of EUDRAGIT® L-100-55.

FIG. 5 illustrates an XRD diffractogram of a 1:2 solid solution of cinacalcet with EUDRAGIT L-100-55.

FIG. 6 illustrates a DSC thermogram of cinacalcet raw material.

FIG. 7 illustrates a DSC thermogram of PVP K-30.

FIG. 8 illustrates a DSC thermogram of a 1:2 solid solution of cinacalcet with PVP K-30.

FIG. 9 illustrates a DSC thermogram of EUDRAGIT® L-100-55.

FIG. 10 illustrates a DSC thermogram of a 1:2 solid solution of cinacalcet with EUDRAGIT L-100-55.

FIG. 11 illustrates a dissolution profile of a solid solution of cinacalcet with PVP (P-00709), of a physical mixture of cinacalcet with PVP (1:3), and of SENSIPAR® in 0.05 N HCl, USP apparatus 2, 37° C., 75 r.p.m.; and

FIG. 12 illustrates a dissolution profile of a solid solution of cinacalcet with PVP (P-00709), of a physical mixture of cinacalcet with PVP (1:3), and of SENSIPAR® in 6 g/L NaH₂PO₄, pH 6, 0.15% SLS, USP apparatus 2, 37° C., 75 r.p.m.

FIG. 13 illustrates a dissolution profile of a 1:2 solid solution of cinacalcet with EUDRAGIT® and of SENSIPAR® in 0.05 N HCl, U.S.P. type 2 apparatus, 37° C., 75 r.p.m.; and in 6 g/L NaH₂PO₄, pH 6, 0.15% SLS, USP apparatus 2, 37° C., 75 r.p.m.

FIG. 14 illustrates a dissolution profile of formulations of solid solutions of cinacalcet in a simulated gastrointestinal environment.

### DETAILED DESCRIPTION OF THE INVENTION

As used herein, unless otherwise defined, “cinacalcet” means cinacalcet free base and pharmaceutically acceptable salts and solvates thereof. Preferably, “cinacalcet” means cinacalcet hydrochloride.

As used herein, unless otherwise defined, “intimate association” or “intimately associated,” when used with respect to a mixture of cinacalcet and at least one carrier, means that the carrier(s) and the cinacalcet interact on the molecular level, there being no easily detectable separate cinacalcet phase. A non-intimate association is, for example, a powder blend, or a compressed powder blend, as it is possible to discern between different phases using, for example, electron microscopy. Another example of a non-intimate association is an emulsion, where separate phases co-exist in the solution, and may be visualized using, for example, a light microscope. An example for an intimate associations is a liquid solution, where there is no way to separate between the carrier and the solute by physical means, or to observe a phase of solute within the carrier.

As used herein, unless otherwise defined, “free drug” means solid particles consisting essentially of cinacalcet that are not in intimate association with a carrier.

As used herein, unless otherwise defined, “non-crystalline” and “not in crystalline form” means a material comprising cinacalcet that does not produce an X-ray powder diffraction pattern having peaks characteristic of crystalline cinacalcet, and that does not exhibit a discernable endotherm in differential scanning calorimetry using heating rates of 2 to 20 degrees per minute.

As used herein, unless otherwise defined, a “solid solution” means a solid, homogenous mixture of at least two components (e.g. cinacalcet and a carrier) wherein the components are interspersed on a molecular level. In solid solutions, the individual physical properties related to the crystalline structure of the components present in lesser amounts, commonly referred to as the solutes, are lost. Presence of the solutes can be detected spectroscopically or by measure of the colligative properties of the solid solution. Even in the solid solutions, some portion of the cinacalcet may come out of solution or remain undissolved in the carrier without departing from the scope of the invention. However, in the solid solutions, at least about 85 percent of the cinacalcet is in solution in the solid solution. Preferably, substantially all, and, most preferably, all, of the cinacalcet is in solution in the solid solution.

As used herein, the term “supercritical fluid” refers to substances at a temperature and pressure above their thermodynamic critical point. A supercritical fluid solution is a solution in which a supercritical fluid is the solvent. Such a substance has unique properties, such as the ability to diffuse through a solid like a gas, and dissolve solids like a liquid. Additionally, it is possible to change the density of such a substance by subtle changes in temperature and/or pressure. Useful supercritical fluids are, for example, carbon dioxide, water, methane, ethane, propane, ethylene, propylene, methanol, ethanol, and acetone. Preferably, the supercritical fluid is selected from the group comprising carbon dioxide, water, and ethanol. More preferably, the supercritical fluid is carbon dioxide.

As used herein, unless otherwise defined, “effective” and “therapeutically effective” amount of a drug or pharmacologically active agent means an amount of the drug or agent that is nontoxic and sufficient to provide the desired effect, e.g., treatment of secondary hyperparathyroidism.

As used herein, unless otherwise defined, “treating,” “treated,” and “treatment” means at least one of the following: reduction in severity and/or frequency of symptoms, elimination of symptoms and/or underlying cause, prevention of the occurrence of symptoms and/or their underlying cause, or improvement or remediation of damage.

The invention encompasses a solid composite comprising cinacalcet and at least one carrier, wherein at least about 85 percent of the cinacalcet is in intimate association with the carrier.

Preferably, the solid composite is a solid solution. Preferably, substantially all of the cinacalcet is in solution in the solid solution. More preferably, all of the cinacalcet is in solution in the solid solution.

Preferably, the solid composite is particulate and has an average particle size larger than about 100 μm, more preferably between 100 and 600 μm.

Preferably at least about 85 percent of the cinacalcet in the solid composite is not in crystalline form. More preferably, the cinacalcet in the solid composite has no detectable crystalline cinacalcet.

The carrier may be any pharmaceutically acceptable inert solid carrier known to one of skill in the art, including, for example, sugars and polymers.

Preferably, the carrier is a hydrophilic polymer or a polymer presenting a pH dependent solubility profile in aqueous media. The hydrophilic polymer may be selected from the group consisting of povidone, poloxamer, hydroxypropyl methylcellulose, polyethylene glycol, copovidone, and amino alkyl methacrylate type A NF.

The polymer presenting a pH dependent solubility profile in aqueous media may be selected from copolymers of methacrylic acid. More preferably, the carrier is povidone or a copolymer of methacrylic acid. Although hydroxypropyl methyl cellulose phthalate, polymethylacrylate, and hydroxypropyl cellulose may be used in the present invention as carriers, other carriers, particularly povidone and copolymers of methacrylic acid, are more preferred in the present invention.

Preferably, the carrier is present in an amount sufficient to maintain at least about 85 percent of the cinacalcet in intimate association with, and, more preferably, in solid solution in, the carrier. One of skill in the art can easily determine by routine experimentation such an amount of carrier. Typically, the drug-to-carrier weight ratio in the solid composites is within a range of about 1:0.5 to about 1:10, preferably about 1:2 to about 1:6.

The invention further encompasses a process for preparing the solid composite comprising: combining cinacalcet, at least one carrier, and at least one liquid solvent to form a solution; and removing the liquid solvent to obtain the solid composite.

The cinacalcet may be prepared by any means known to one of skill in the art. It will be appreciated that while preferably at least about 85 percent of the cinacalcet in the solid composites of the present invention is not in crystalline form, cinacalcet in any form (e.g. crystalline or amorphous) may be used to prepare the solid composites.

Liquid solvents suitable for preparing the solid composite include organic solvents capable of dissolving at least about 85 percent of the cinacalcet and substantially all of the carrier. Preferably, the liquid solvent is capable of dissolving at least about 85 percent of the cinacalcet and at least about 85 percent of the carrier. More preferably, the liquid solvent is capable of dissolving substantially all of the cinacalcet and carrier. Most preferably, the liquid solvent is capable of dissolving all of the cinacalcet and carrier. Preferably, the liquid solvent is one in which cinacalcet has a solubility of at least about 5 mg of cinacalcet per 1 ml solvent at 25° C.

Examples of suitable liquid solvents include, but are not limited to, at least one of lower aliphatic alcohols and C₃₋₈ ketones. “Lower aliphatic alcohols” as used herein means organic compounds having the general structure R—OH, wherein R is a linear or branched C₁₋₆ alkyl group. Preferred lower aliphatic alcohols include methanol, ethanol, isopropyl alcohol (“IPA”), and butanol. Preferred C₃₋₈ ketones include acetone, methylisobutyl ketone (“MIBK”) and methylethyl ketone (“MEK”). More preferred liquid solvents are ethanol, acetone, isopropyl alcohol, and mixtures thereof. Most preferably, the liquid solvent is ethanol or mostly ethanol in combination with one or more of the above solvents.

The combining step may include mixing the liquid solvent with cinacalcet and at least one carrier in any order. The cinacalcet, carrier, and liquid solvent may be mixed using any suitable mixing method known to one of skill in the art, such as by using magnetic stirrers, mixer stirrers, shakers, or sonification.

Preferably, at least about 85 percent of the cinacalcet and a majority of the carrier are in solution in the at least one liquid solvent. More preferably, at least about 85 percent of the cinacalcet and about 85 percent of the carrier are in solution in the at least one liquid solvent. Even more preferably, substantially all of the cinacalcet and carrier are in solution in the at least one solvent. In particularly preferred embodiments, all of the cinacalcet and carrier are in solution in the at least one solvent.

The step of removing the liquid solvent may be performed by any method known to one of skill in the art. Preferably, the liquid solvent is removed by evaporation. More preferably, the liquid solvent is removed by evaporation under vacuum, by fluidized bed drying, or by spray-drying. “Spray-drying” broadly refers to processes involving breaking up liquid mixtures into small droplets (atomization) and rapidly removing solvent from the mixture. In a typical spray-drying apparatus, there is a strong driving force for evaporation of solvent from the droplets, which may be provided by providing a heated drying gas. Spray-drying processes and equipment are described in Perry's Chemical Engineer's Handbook, pp. 20-54 to 20-57 (6th ed. 1984). The obtained solid composite may optionally be further dried.

Preferably, the obtained solid composite is in the form of a solid solution, wherein substantially all, most preferably all, of the cinacalcet is in solid solution in the carrier.

Optionally, the process further comprises the addition of at least one pharmaceutically acceptable excipient. Suitable pharmaceutically acceptable excipients include, for example, surfactants such as sodium lauryl sulfate.

The pharmaceutically acceptable excipient may be combined with the cinacalcet, carrier, and liquid solvent in step a), or may be added to the obtained solid composite of step b) after removal of the liquid solvent.

Alternatively, the pharmaceutically acceptable excipient may be added during removal of the liquid solvent in step b), by, for example, spraying the solution containing the cinacalcet, carrier and solvent onto a fluidized bed of the excipient while drying. As a result, the solid composite will be formed on the excipient. In some cases, this process will prove advantageous because it provides a solid composite with greater surface area, which can aid in dissolution in aqueous media upon administration to a patient.

The invention further encompasses a process for preparing the solid composite using supercritical fluid technology. The process may comprise: dissolving cinacalcet and at least one carrier in a supercritical fluid; and removing the supercritical fluid by evaporation. Preferably, the evaporation is accomplished under reduced pressure or by adjusting the temperature of the solution to a temperature at which the supercritical fluid becomes a gas. Alternatively, the process may comprise: dissolving cinacalcet and at least one carrier in an organic or inorganic solvent to form a liquid solution; adding a supercritical fluid (anti-solvent) to induce precipitation of a mixture of the cinacalcet and the carrier; and removing the solvent and the supercritical fluid by evaporation. Preferably, the evaporation is accomplished under reduced pressure or by adjusting the temperature of the solution.

The invention further encompasses a process for preparing the solid composite comprising: combining cinacalcet and at least one carrier to form a mixture; heating the mixture to a temperature at which both the cinacalcet and the carrier melt to form a fusion product; and cooling the fusion product in a manner that does not allow for re-crystallization of the cinacalcet out of the fusion product.

The invention further encompasses a process for preparing the solid composite comprising: combining cinacalcet and at least one carrier to form a mixture, heating the mixture to control its viscosity; and feeding the heated mixture through a hot melt extrusion system.

It would be appreciated by one of skill in the art that the above-described solid composites reduce the health risks involved in handling active pharmaceutical ingredients in particulate form, and specifically micronized particles.

In addition, because of the enhanced solubility of the solid composites relative to the active ingredient alone or in a physical mixture with the carrier, as described below, a larger particle size of the solid composite particles may be used in the formulation without having an adverse effect on the dissolution profile of the cinacalcet.

Another method to improve the safety of the products is by using a bi-modal distribution of particle sizes of the cinacalcet, wherein the population of large particle sizes, above 70 μm, avoids the need for micronization of the cinacalcet, and thus reduces production of hazardous dust. The population of small particle sizes, smaller than about 5 μm, preferably less than 2 μm, and even more preferably smaller than 1 μm, is prepared using a high pressure homogenizer. This method involves micronizing the particles in a liquid medium, thus avoiding production and dispersion of dust particles of the active ingredient.

The invention further encompasses pharmaceutical formulations comprising a solid composite comprising cinacalcet and at least one carrier, and at least one pharmaceutically acceptable excipient.

The pharmaceutical formulation may contain additional cinacalcet, meaning free drug cinacalcet in addition to the cinacalcet in the solid composite, thus providing the ability to manipulate the dissolution characteristics of the formulation.

The amount of the solid composite used in the pharmaceutical formulation is preferably an amount that provides a therapeutically effective amount of cinacalcet. It will be appreciated that the amount of solid composite used will differ according to the cinacalcet: carrier ratio in the particles.

Preferably, the pharmaceutical formulation comprises: (a) from about 10 percent to about 40 percent by weight of a calcium receptor-active compound, such as cinacalcet; (b) from about 10 percent to about 50 percent of at least one binder, which may serve as the carrier or “solid solvent” of the solid composite; (c) from about 15 percent to about 45 percent by weight of at least one diluent; and (e) from about 10 percent to about 40 percent of at least one disintegrant; wherein the percentage by weight is relative to the total weight of the formulation.

The formulation may further comprise about 0.5 percent to about 5 percent by weight relative to the total weight of the formulation of at least one glidant or lubricant and about 1 percent to 6 percent by weight of at least one coating material. It is understood by one of skill in the art that one or more inactive ingredients can act in more than one capacity for example; the same material may function as both a diluent and a disintegrant. The formulation may further comprise a surfactant.

The pharmaceutical formulation can be processed into, for example, a unit dosage form. In particular, the pharmaceutical formulation can be formulated into oral solid dosage forms such as capsules, tablets, or gel-caps.

The solid composites and pharmaceutical formulation including them preferably allow for the rapid absorption and onset of the calcium receptor-active compound cinacalcet in a mammal.

The invention further encompasses an immediate release formulation of a calcium receptor-active compound, such as cinacalcet, wherein at least about 80 percent of the calcium receptor-active compound is released from the formulation within about 30 minutes in 0.05 N HCl in a U.S.P. type 2 apparatus at a temperature of about 37° C. and at a rotation speed of about 75 r.p.m.

The invention further encompasses a controlled release formulation of a calcium receptor-active compound, such as cinacalcet, wherein the majority of the calcium receptor-active compound is released in the intestine, where the pH is slightly acidic to neutral, rather than in the stomach, where the pH is acidic.

When the controlled release formulation is exposed to a simulated gastric environment for a period of about 30 minutes, followed by exposure to a simulated intestinal environment, more than about 50 percent of the calcium receptor-active compound is released from the formulation during about the first 30 minutes of exposure and not less than about 70 percent of the calcium receptor-active compound is released from the formulation during about the first 90 minutes of exposure.

Preferably, at least about 50 percent of the calcium receptor-active compound is released within about the first 60 minutes of exposure, i.e., 30 minutes after the change in environment.

Preferably, not less than about 80 percent of the calcium receptor-active compound is released from the formulation during about the first 90 minutes of exposure. More preferably, not less than about 90 percent of the calcium receptor-active compound is released from the formulation during about the first 90 minutes of exposure.

The simulated gastric environment is 800 ml of 0.05 N HCl in a U.S.P. type 2 apparatus at a temperature of about 37° C. and at a rotation speed of about 75 r.p.m. The simulated intestinal environment is 6 g/L NaH₂PO₄, pH 6, 0.15% sodium lauryl sulfate in a U.S.P. type 2 apparatus at a temperature of about 37° C. and at a rotation speed of about 75 r.p.m. These environments simulate the passage of the dosage forms through the gastrointestinal pathway.

The invention further encompasses a method of treatment comprising administering the pharmaceutical formulation to a mammal. Preferably, the mammal is a human. Preferably, the pharmaceutical formulation comprises cinacalcet and is administered to treat secondary hyperparathyroidism, which is the approved use for SENSIPAR®. As discussed above, the method of “treating” secondary hyperparathyroidism described herein encompasses both prevention of the disorder in a predisposed individual and treatment of the disorder in a clinically symptomatic individual.

The amount of calcium receptor-active compound administered and the dosing regimen used, will depend on the particular drug selected, the age and general condition of the subject being treated, the severity of the subject's condition, and the judgment of the prescribing physician.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

In the following examples, a rotary evaporator equipped with a vacuum pump, was used to remove the solvent from the liquid solutions. The liquid solutions were heated to about 50° C. using a water bath during rotary evaporation.

In the following examples, dissolution profiles were determined in a dissolution vessel using a U.S.P. type 2 apparatus (paddles) in a simulated gastrointestinal environment under conditions described in Table 1. See U.S. Pharamcopeia, pp. 2155-2156 (26th ed. 2003). Samples were analyzed on-line by a UV detector.

TABLE 1
Dissolution conditions.

| | |
|---|---|
| Medium 1 | 0.05 N HCl, pH = 1.3 |
| Medium 2 | 0.15% aqueous solution sodium lauryl sulfate and |
| | 6 g/L NaH₂PO_{4.} pH = ~6.0 |
| Volume | 900 ml |
| Temperature | 75° C. |
| Speed | 50 RPM |
| Sampling points: | 5, 10, 30, 50, 80, 120 and (optionally) 160 minutes |

### Example 1

### Cinacalcet Solid Solutions with Povidone

a) Cinacalcet HCl:Povidone in 1:2 Weight Ratio

1 g of cinacalcet HCl per 2 g of povidone (PVP K-30) were ground together using a mortar and pestle. The resulting mixture was completely dissolved in ethanol in a round-bottom flask. The ethanol was then removed from the solution using a rotary evaporator under vacuum, and heating the solution to 50° C., until dry solid flakes formed on the flask. The dry solid was then collected.

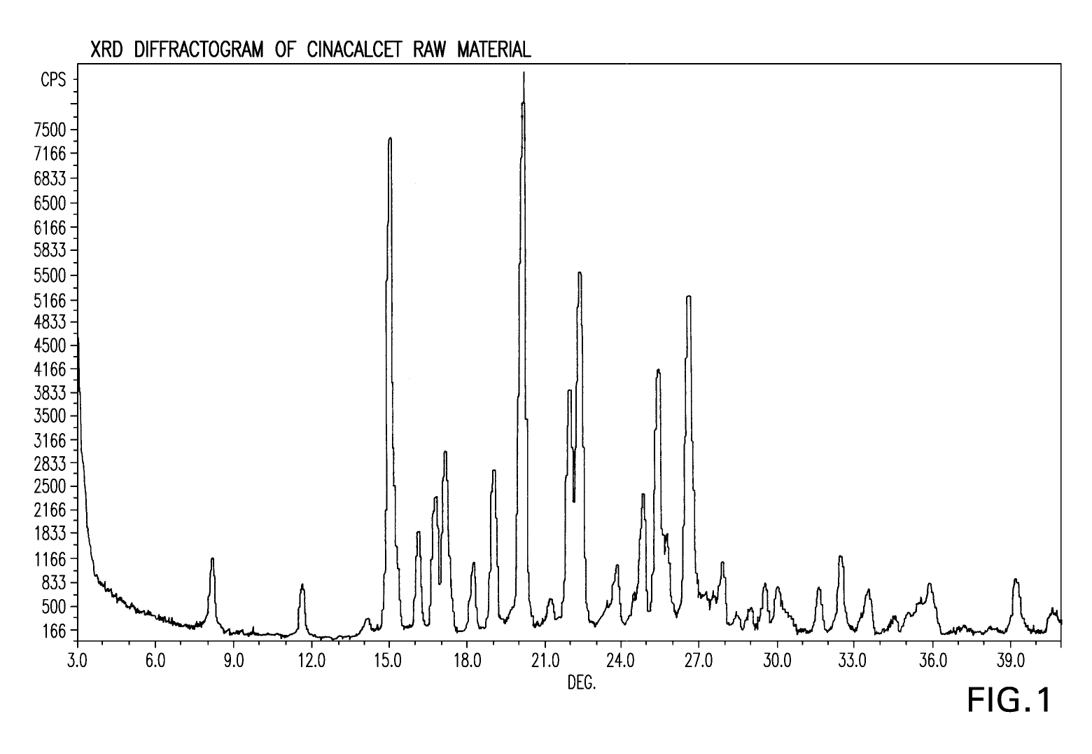

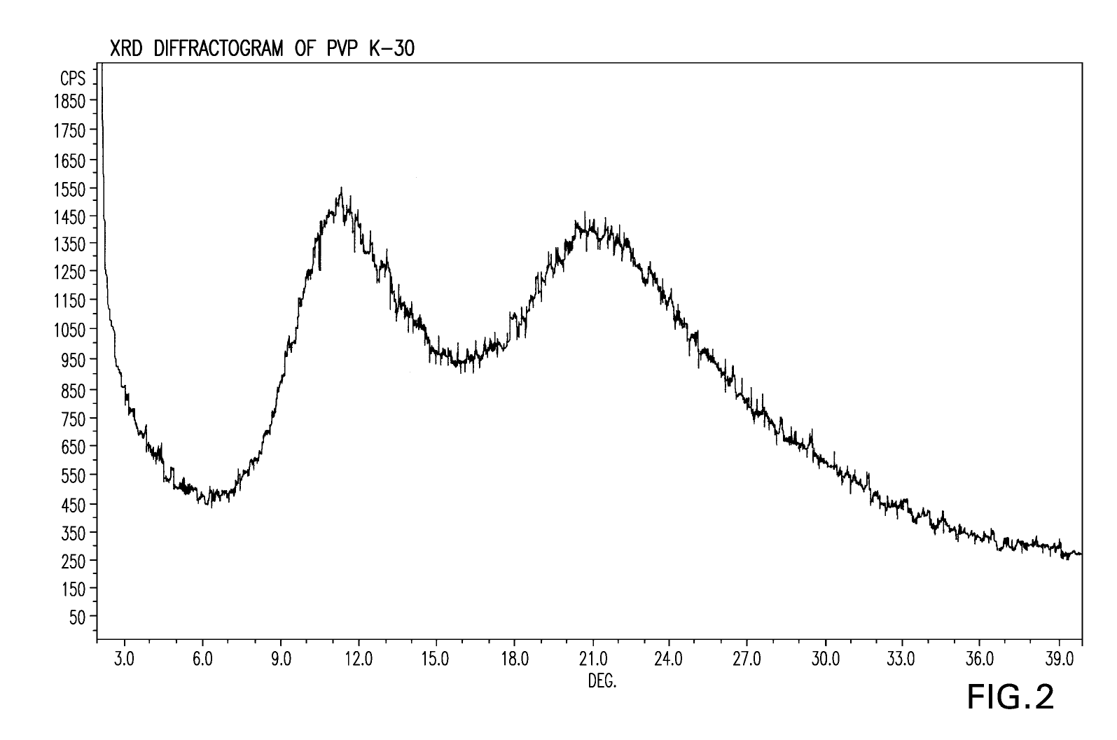

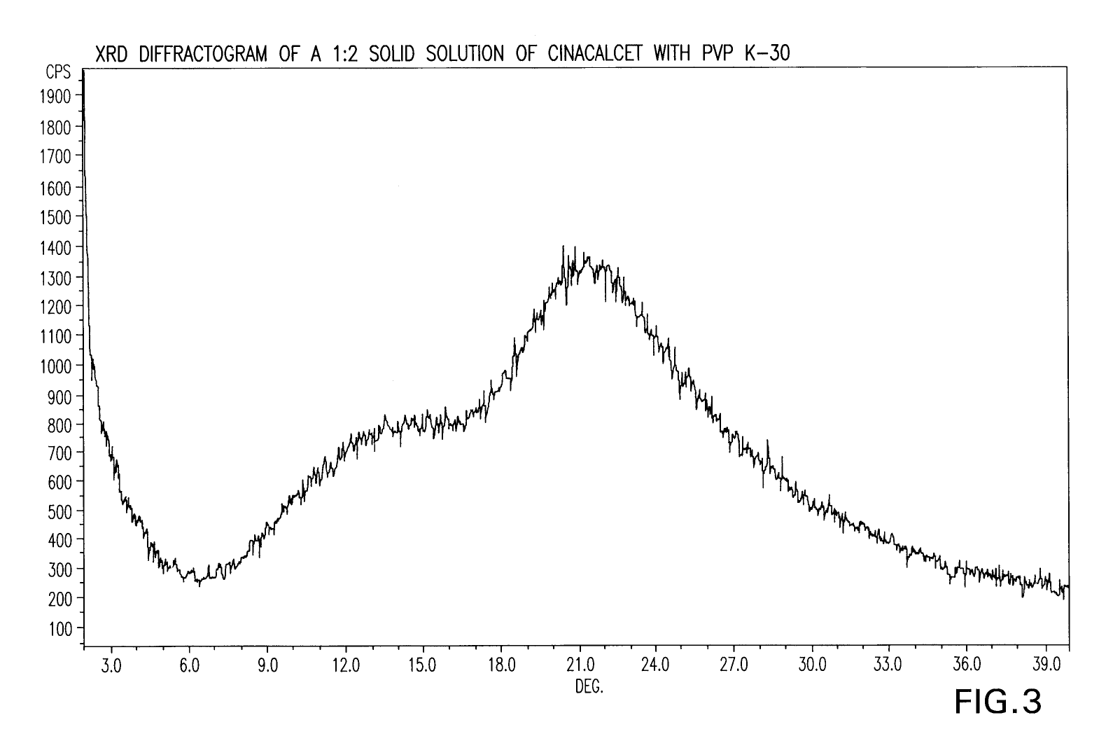

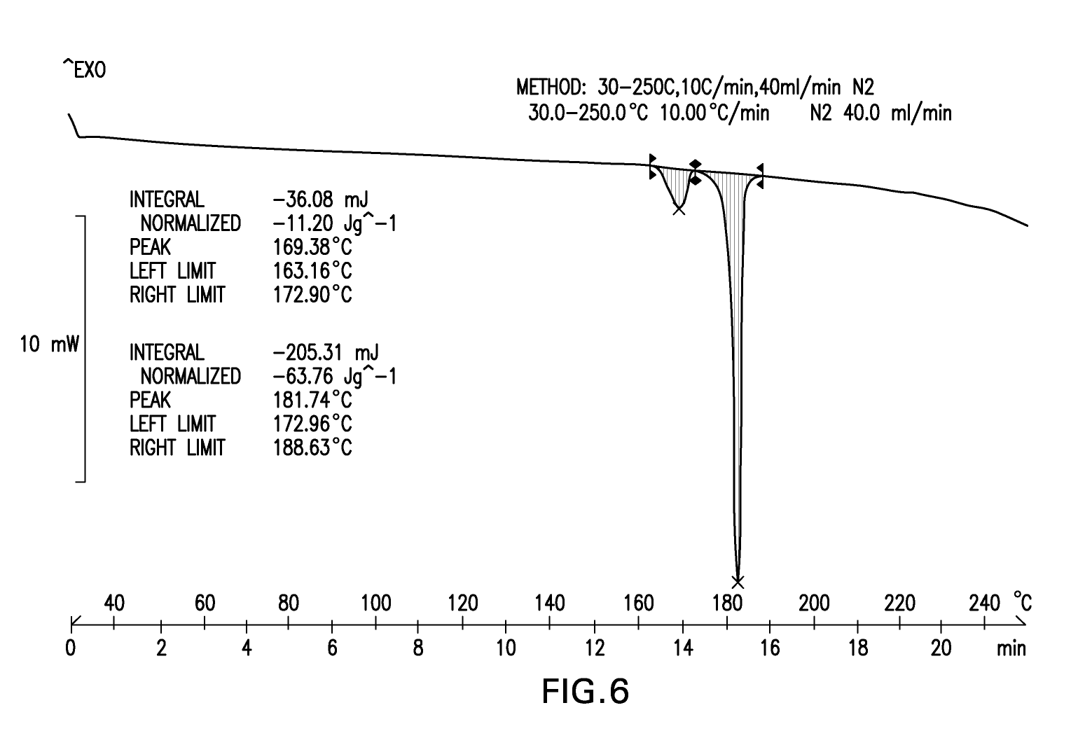

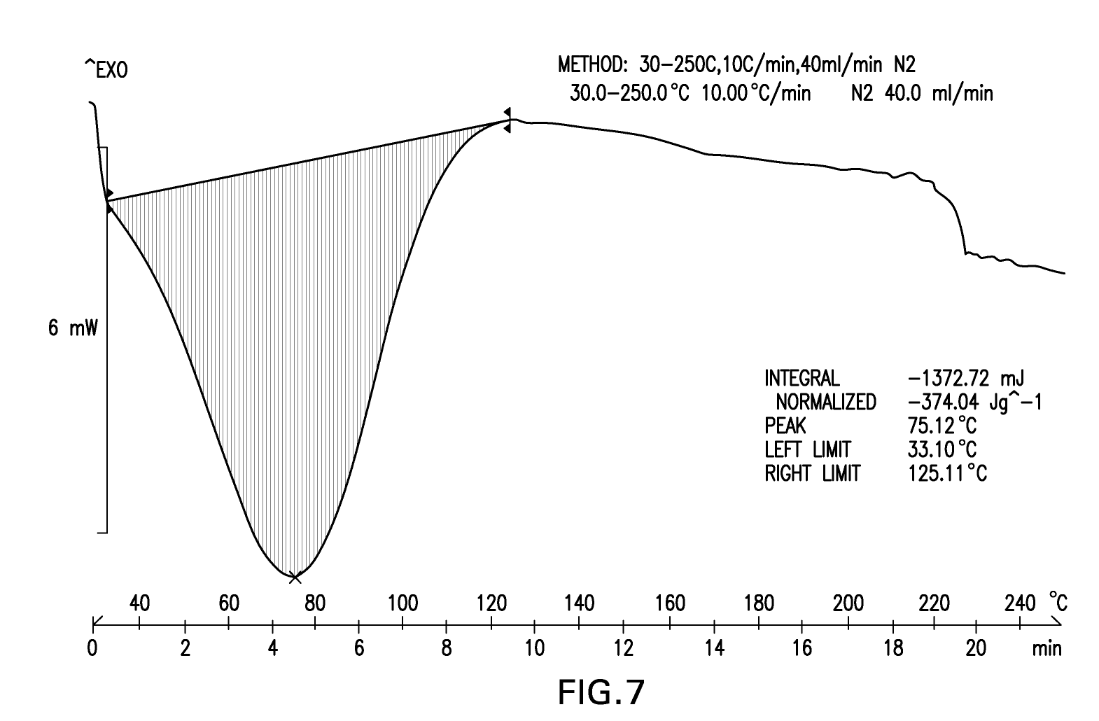

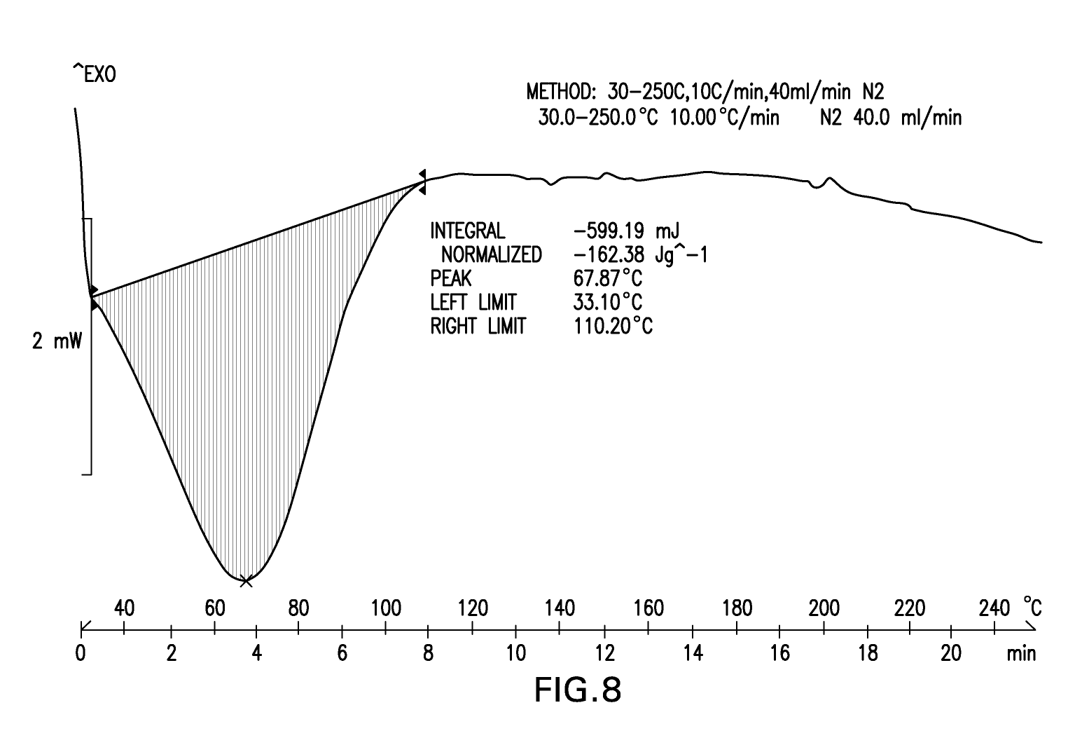

X-ray diffraction (“XRD”) and differential scanning calorimetry (“DSC”) were performed on the dry solid and compared to the XRD and DSC for the cinacalcet and povidone alone. The XRD and DSC for the dry solid are illustrated in FIGS. 3 and 8, respectively. The XRD and DSC for the cinacalcet are illustrated in FIGS. 1 and 6, respectively. The XRD and DSC for the povidone are illustrated in FIGS. 2 and 7, respectively.

b) Cinacalcet HCl:Povidone in 1:3 Weight Ratio

1 g of cinacalcet HCl per 3 g of povidone (PVP K-30) were ground together using a mortar and pestle. The resulting mixture was completely dissolved in ethanol in a round-bottom flask. The ethanol was then removed from the solution using a rotary evaporator under vacuum, and heating the solution to 50° C., until dry solid flakes formed on the flask.

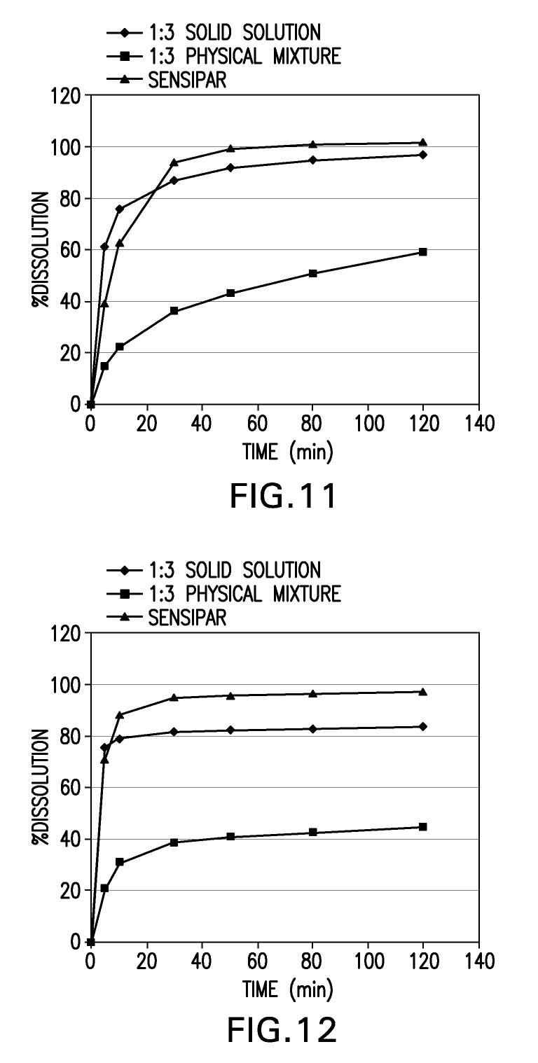

A sample of the dry solid was collected and its dissolution profile determined according to the conditions in Table 1. The dissolution profile of the solid was compared with the dissolution profile of a commercial version of cinacalcet HCl tablets (SENSIPAR®, 30 mg) and with a simple mixture of cinacalcet HCl (average length of the needle-shaped crystals was ˜20 micron) with povidone. The results of dissolution testing are illustrated in FIGS. 11 and 12.

The amount of cinacalcet dissolved at the 30 minute time point demonstrates that cinacalcet in a solid solution form has a solubility greater than cinacalcet raw material in a physical mixture with lactose or starch. The percent of cinacalcet dissolved after 5 minutes demonstrates the greater dissolution rate of cinacalcet in a solid solution form, as compared with both SENSIPAR® and a physical mixture of particles with an average length of 20 μm with starch.

### Example 2

### Cinacalcet Solid Solution with EUDRAGIT® L-100-55 in 1:2 Weight Ratio

1 g of cinacalcet was dissolved in 10 ml of ethanol to form a first solution. 2 g of EUDRAGIT® L-100-55 was dissolved in about 15 ml of ethanol to form a second solution. The two solutions were then combined, and ethanol was evaporated from the combined solution using a rotary evaporator to obtain dry solid flakes. The dry solid was then collected.

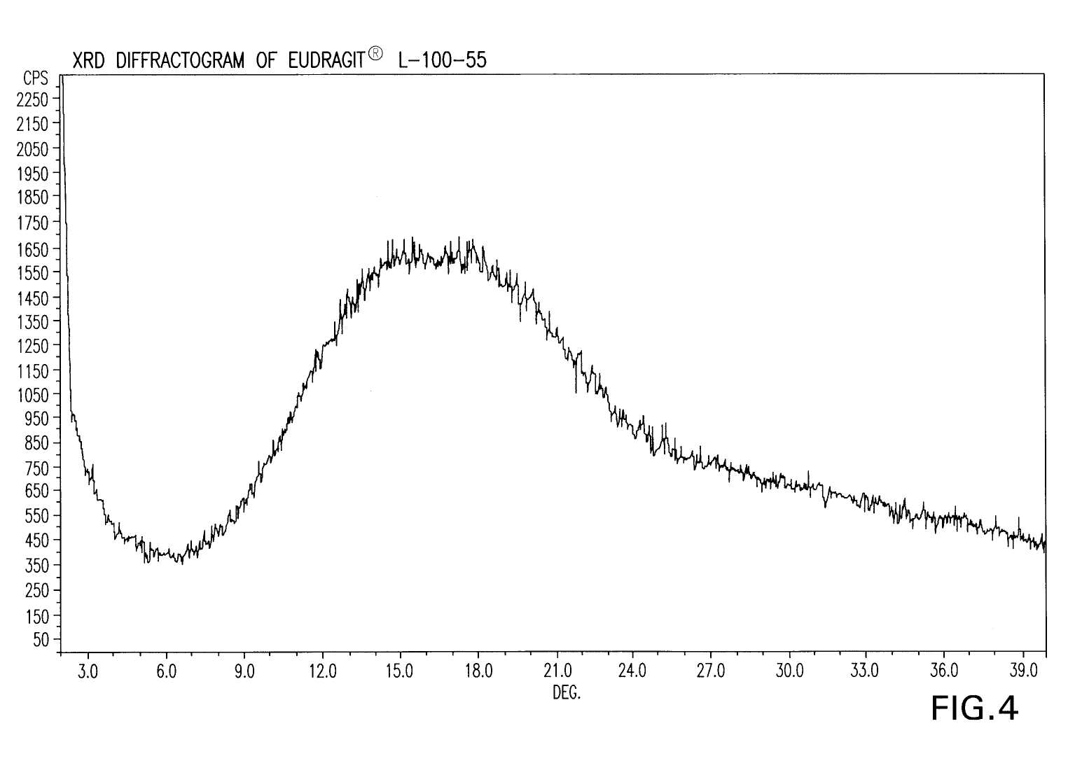

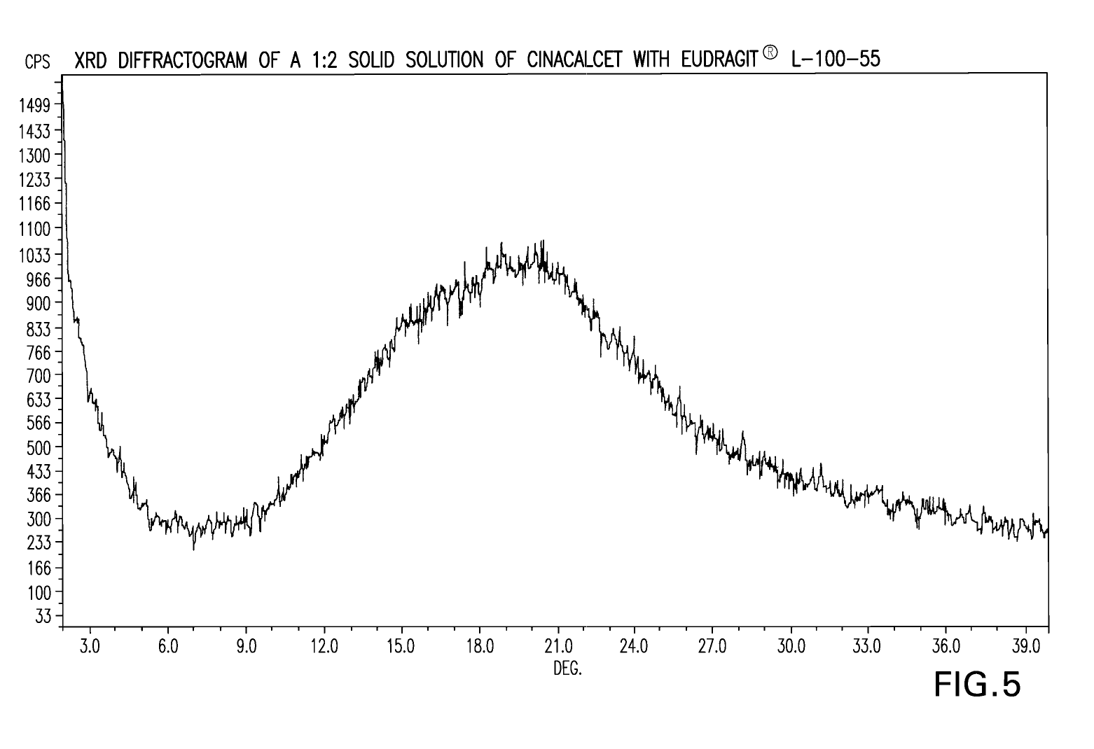

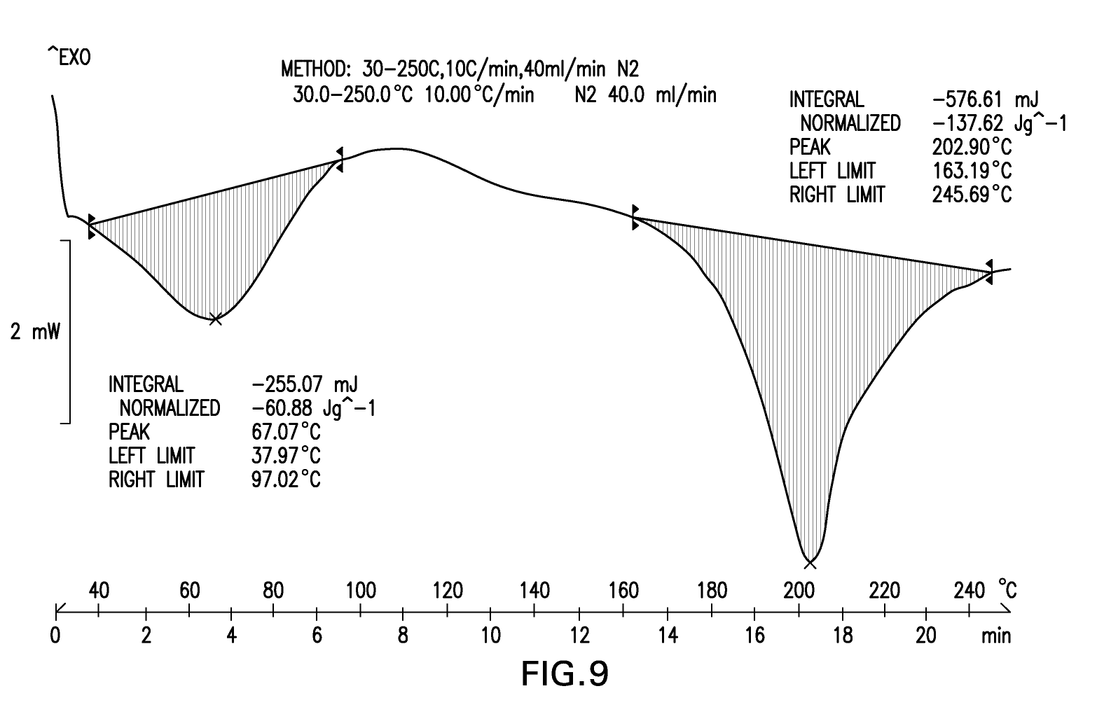

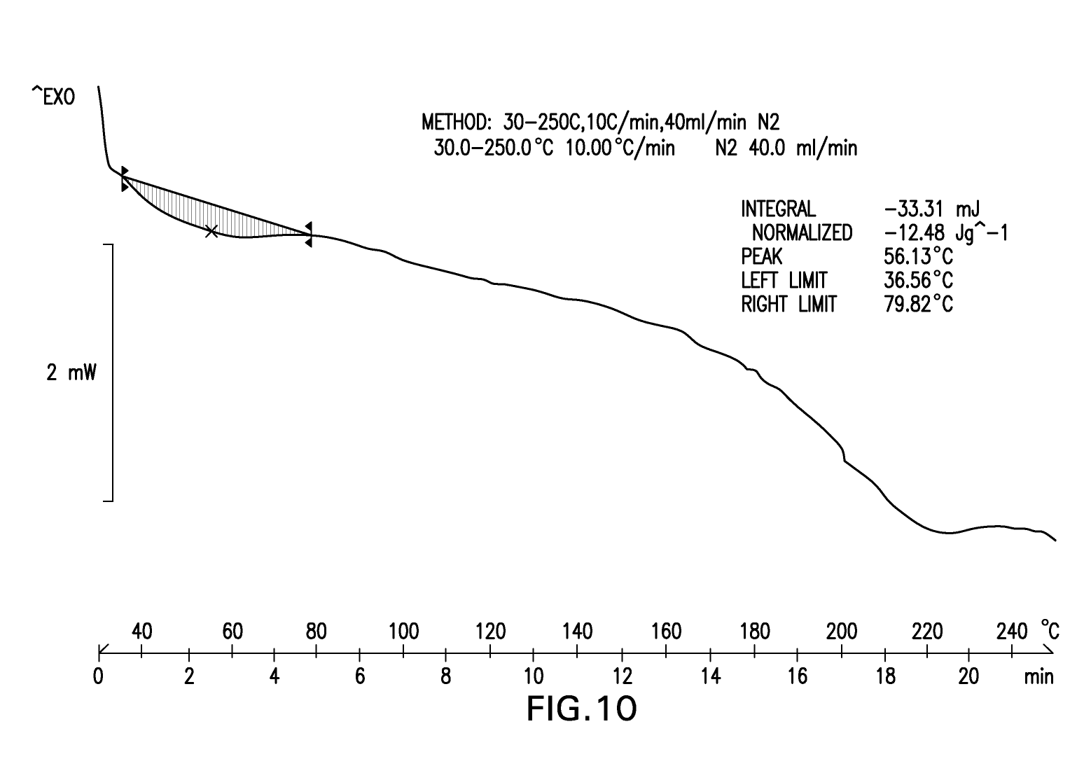

XRD and DSC were performed on the dry solid and compared to the XRD and DSC for the cinacalcet and EUDRAGIT® L-100-55 alone. The XRD and DSC for the dry solid are illustrated in FIGS. 5 and 10, respectively. The XRD and DSC for the cinacalcet are illustrated in FIGS. 1 and 6, respectively. The XRD and DSC for the EUDRAGIT® L-100-55 are illustrated in FIGS. 4 and 9, respectively.

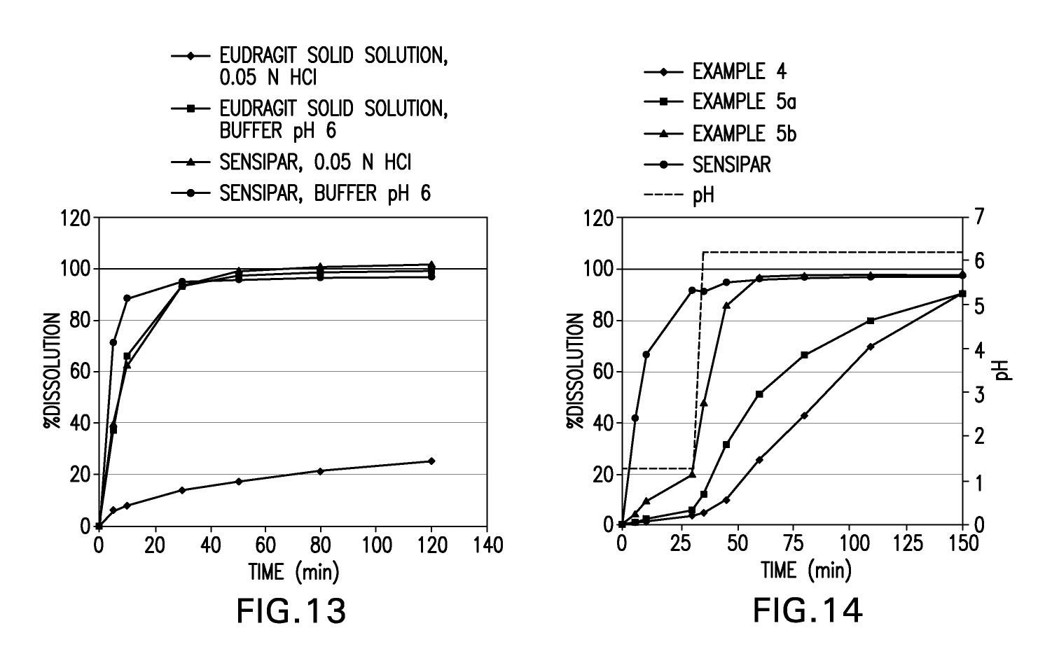

A sample of the dry solid was collected and its dissolution profile determined according to the conditions in Table 1. The dissolution profile of the solid was compared with the dissolution profile of a commercial version of cinacalcet HCl tablets (SENSIPAR®, 30 mg) in a simulated gastrointestinal environment. The results of dissolution testing are shown in FIG. 13.

As illustrated in FIG. 13, in the simulated gastric environment of 0.05N HCl, cinacalcet was released more quickly from the SENSIPAR® tablets than from the solid solution of cinacalcet and EUDRAGIT® L-100-55. For example, about 95 percent of the cinacalcet was released from the SENSIPAR® tablets during about the first 30 minutes of exposure to the 0.05N HCl, while only about 15 percent of the cinacalcet was released from the solid solution of cinacalcet and EUDRAGIT® L-100-55 the under the same conditions. In the more neutral simulated intestinal environment of buffer at pH 6, however, about 95 percent of the cinacalcet was released from both the SENSIPAR® tablets and from the solid solution of cinacalcet and EUDRAGIT® L-100-55 during about the first 30 minutes of exposure.

### Example 3

### Formulation Including Cinacalcet Solid Solution with Povidone

Prepare a formulation of cinacalcet, with a target amount of 90 mg of cinacalcet per tablet, having the following composition:

TABLE 2
Cinacalcet formulation prepared in Example 3.

| | | Amount |
|---|---|---|
| | Weight % | (mg/ |
| Ingredient | (w/w) | tablet) |
| Cinacalcet HCl | 15.88 | 99.18 |
| Povidone (PVP K-30) | 31.76 | 198.36 |
| Microcrystalline cellulose (AVICEL ® PH102) | 32.02 | 200.0 |
| Crospovidone | 9.61 | 60.0 |
| Sodium Carboxymethylcellulose (AC-DI-SOL ®) | 9.61 | 60.0 |
| Magnesium stearate | 1.12 | 7.0 |
| Core Tablet | 100.00 | 624.54 |

Combine and mix cinacalcet HCl, povidone, microcrystalline cellulose, crospovidone, and sodium carboxymethylcellulose. Then, add magnesium stearate to the mixture and press the mixture into tablets. Measure the dissolution profile of the tablets according to the procedure described in Example 6.

### Example 4

### Formulation Including Cinacalcet Solid Solution with Povidone

A solid solution of cinacalcet with povidone (1:2 weight ratio) prepared according to Example 1a was passed through a sieve equipped with a 30 mesh (−600 micron aperture) screen on top of a 50 mesh screen (300 micron aperture).

The sample, which passed the 50 mesh screen, is estimated to have a particle size distribution wherein approximately 100 percent of the particles are less than 300 microns in size and an average particle size around 100 μm. This sample was used to prepare a pharmaceutical formulation of cinacalcet, with a target amount of 90 mg of cinacalcet per tablet, having the following composition:

TABLE 3
Cinacalcet formulation prepared in Example 4.

| | | Amount |
|---|---|---|
| | Weight % | (mg/ |
| Ingredient | (w/w) | tablet) |
| Cinacalcet HCl | 16.68 | 99.18 |
| Povidone (PVP K-30) | 33.36 | 198.36 |
| Microcrystalline cellulose (AVICEL ® PH102) | 25.24 | 150.0 |
| Crospovidone | 10.09 | 60.0 |
| Sodium Carboxymethylcellulose (AC-DI-SOL ®) | 10.09 | 60.0 |
| EUDRAGIT ® L-100-55 | 3.36 | 20.0 |
| Magnesium stearate | 1.18 | 7.0 |
| Core Tablet | 100.00 | 594.54 |

The solid solution of cinacalcet with povidone was dry-mixed with microcrystalline cellulose, crospovidone, and sodium carboxymethylcellulose, followed by granulation with a 20 percent to 40 percent ethanolic solution of EUDRAGIT® L-100-55. The resulting granulate was then dried in a vacuum oven at 50° C. The granulate was then passed through an 8 mesh sieve (˜2.4 mm), followed by an 18 mesh sieve (˜1 mm). Magnesium stearate was then added to the granulate to form a lubricated blend, and the final lubricated blend was compressed into tablets using a manual press.

### Example 5

### Formulation Including Cinacalcet Solid Solution with Eudragit L-100-55

A solid solution of cinacalcet in EUDRAGIT® L-100-55 (1:2 weight ratio) prepared according to Example 2 was passed through a sieve equipped with a 30 mesh screen on top of a 50 mesh screen.

a) Cinacalcet HCl:EUDRAGIT® L-100-55 with Average Particle Size of ˜400 μm

The solid composite collected on top of the 50 mesh screen (having a particle size between 300 μm and 600 μm) was used in a pharmaceutical formulation (average particle size estimated to be ˜400 μm).

b) Cinacalcet HCl:EUDRAGIT® L-100-55 with Average Particle Size of ˜100 μm

The solid composite which passed through the 50 mesh screen (having a particle size distribution wherein 100 percent of the particles are less than 300 μm in size) was used in another pharmaceutical formulation (average particle size estimated to be ˜100 μm).

Each solid composite (5a and 5b) was used to prepare a pharmaceutical formulation of cinacalcet, with a target amount of 90 mg of cinacalcet per tablet, having the following composition:

TABLE 4
Cinacalcet formulation prepared in Example 5.

| | | Amount |
|---|---|---|
| | Weight % | (mg/ |
| Ingredient | (w/w) | tablet) |
| Cinacalcet HCl | 15.88 | 99.18 |
| EUDRAGIT ® L-100-55 | 31.76 | 198.36 |
| Microcrystalline cellulose (AVICEL ® PH102) | 32.02 | 200.0 |
| Crospovidone | 9.61 | 60.0 |
| Sodium Carboxymethylcellulose (AC-DI-SOL ®) | 9.61 | 60.0 |
| Magnesium stearate | 1.12 | 7.0 |
| Core Tablet | 100.00 | 624.54 |

The solid solution of cinacalcet with EUDRAGIT® L-100-55 was mixed with microcrystalline cellulose, crospovidone, and sodium carboxymethylcellulose, followed by granulation. Magnesium stearate was then added to the granulate to form a lubricated blend, and the final lubricated blend was compressed into tablets using a manual press.

### Example 6

### Dissolution Profile of Tablet Formulations of Cinacalcet

The dissolution profiles of the formulations prepared in Examples 4 and 5 were measured with a U.S.P. type 2 apparatus at a temperature of about 37° C., and at a rotation speed of about 75 r.p.m. The dissolution profiles are described in Table 5 and illustrated in FIG. 14. Percentage of dissolution was adjusted to change in medium volume.

The dissolution profile was measured in a complex medium designed to imitate physiological conditions in the gastrointestinal tract. During the first 30 minutes of dissolution the medium was 800 ml 0.05N HCl, which was then neutralized with 120 ml of a neutralizing buffer (50 g/l NaH₂PO₄ adjusted to pH 6, to which were added 35 ml/l 10 N NaOH, and 11.25 g/l SLS) for a final volume of 920 ml.

TABLE 5
Dissolution profiles for the formulations
prepared in Examples 4, 5a, and 5b.

| | % Dissolution | | | |
|---|---|---|---|---|
| | | | | SENSIPAR ® |
| Time (min) | Example 4 | Example 5a | Example 5b | (90 mg) |
| 5 | 0.8 | 0.9 | 4.9 | 41.5 |
| 10 | 1.5 | 2.1 | 9.6 | 66.3 |
| 30 | 3.5 | 5.7 | 19.8 | 91.3 |
| 35 | 4.5 | 12.3 | 47.3 | 90.7 |
| 45 | 10.0 | 30.7 | 86.2 | 94.3 |
| 60 | 25.1 | 50.4 | 96.4 | 95.6 |
| 80 | 42.1 | 65.9 | 97.3 | 96.2 |
| 110 | 69.7 | 79.5 | 97.5 | 96.7 |
| 150 | 90.0 | 89.9 | 97.7 | 96.9 |

As illustrated in Table 5, in the simulated gastric environment of 0.05N HCl, cinacalcet was released more quickly from the SENSIPAR® tablets than from the solid solution of cinacalcet and EUDRAGIT® L-100-55. 91.3 percent of the cinacalcet was released from the SENSIPAR® tablets during the first 30 minutes of exposure to the 0.05N HCl, while only 3.5 to 19.8 percent of the cinacalcet was released from the solid solution of cinacalcet and povidone the under the same conditions. In the more neutral simulated intestinal environment of buffer at pH 6, however, 90 percent to 97.7 percent of the cinacalcet was released from both the SENSIPAR® tablets and the solid solution of cinacalcet and povidone during the first 150 minutes. Thus, the solid solution allows for the controlled release of cinacalcet in an environment more neutral than the gastric environment.

### Example 7

### Method for Improving the Safety of Cinacalcet Production—Preparation of Sub-Micron Scale Population of Particles

A suspension of cinacalcet hydrochloride in water is placed in a Microfluidics M-110Y Laboratory Microfluidizer Processor, and processed until the D₅₀ particle size is less than about 1 μm. This population is mixed with a population of particles having a D₅₀ of more than about 100 μm, so that the overall particle size D₅₀ is larger than about 70 μm. This mixture is then used in a formulation, and the dissolution profile is examined.

### Example 8

### Preparation of Cinacalcet Solid Solutions Using Supercritical Fluid Technology

A solution of cinacalcet in absolute ethanol, also containing PVP K-30, is introduced in the particle formation vessel of a supercritical apparatus containing supercritical carbon dioxide at an appropriate flow through the inner nozzle passage. Supercritical carbon dioxide is introduced at an appropriate flow through the outer nozzle passage. A solid fluffy substance is formed, and the pressure lowered to remove the carbon dioxide and ethanol.

What is claimed is:

1. A method for preparing a solid composite, comprising:
combining cinacalcet, at least one carrier, and at least one liquid solvent to form a solution; and
removing the solvent to obtain a solid composite of the cinacalcet and the at least one carrier.

2. The method of claim 1, wherein the solid composite is a solid solution.

3. The method of claim 1, wherein the carrier is selected from the group consisting of povidone, poloxamer, hydroxypropyl methylcellulose, polyethylene glycol, copovidone, copolymers of methacrylate, and copolymers of methacrylic acid.

4. The method of claim 1, further comprising removing solvent by evaporation.

5. The method of claim 1, further comprising dissolving the cinacalcet and at least one carrier in an organic or inorganic solvent to form the solution;
adding a supercritical fluid to induce precipitation of a mixture of the cinacalcet and the carrier; and
removing the solvent and the supercritical fluid by evaporation.

6. The method of claim 1, wherein the solvent is a supercritical fluid.

7. The method of claim 6, wherein the supercritical fluid is selected from the group consisting of carbon dioxide, water, methane, ethane, propane, ethylene, propylene, methanol, ethanol, and acetone.

8. The method of claim 6, further comprising removing the supercritical fluid by reducing pressure.

9. A method for preparing a solid composite, comprising:
combining cinacalcet and at least one carrier to form a mixture;
heating the mixture to a temperature at which both the cinacalcet and the carrier melt to form a fusion product; and
cooling the fusion product in a manner that does not allow crystallization of the cinacalcet out of the fusion product.

10. The method of claim 9, further comprising combining the cinacalcet and at least one carrier to form a mixture;
heating the mixture to control its viscosity; and
feeding the heated mixture through a hot melt extrusion system.
